(19) United States
(12) Patent Application Publication
(10) Pub. No.: US 2024/0115139 A1
(43) Pub. Date: Apr. 11, 2024

(54) METHOD AND A SENSOR FOR DETERMINING A CONCENTRATION OF A TARGET GAS IN THE BLOOD OF AN ANIMATE BEING

(71) Applicants: Fraunhofer-Gesellschaft zur Förderung der angewandten Forschung e.V., München (DE); Albert-Ludwigs-Universität Freiburg, Freiburg (DE)

(72) Inventors: Johannes KAPP, Freiburg (DE); Katrin SCHMITT, Freiburg (DE); Christian WEBER, Freiburg (DE); Jürgen WÖLLENSTEIN, Freiburg (DE); Hassan YASSINE, Freiburg (DE)

(73) Assignees: Albert-Ludwigs-Universität Freiburg, Freiburg (DE); Fraunhofer-Gesellschaft zur Förderung der angewandten Forschung e.V., München (DE)

(21) Appl. No.: 18/277,161

(22) PCT Filed: Jan. 28, 2022
(86) PCT No.: PCT/EP2022/051999

§ 371 Date: Aug. 14, 2023

(30) Foreign Application Priority Data
Feb. 19, 2021 (EP) 21158069.1

(51) Int. Cl. A61B 5/00; A61B 5/145

(52) U.S. Cl. CPC A61B 5/0095; A61B 5/14542; A61B 2562/0204

(57) ABSTRACT

A method for determining a concentration of a target gas in blood of a human or animal animate being. The method includes the steps of: generating electromagnetic excitation radiation having a carrier frequency, wherein the carrier frequency is selected such that the target gas and an absorbing gas absorb the excitation radiation, modulating an amplitude or the carrier frequency of the excitation radiation with a modulation frequency, illuminating an absorption path superficial to a skin of the animate being with the excitation radiation, generating a sound wave by an absorption of the excitation radiation in the absorption gas, and detecting at least one of an amplitude and a phase of the sound wave as a measure of a concentration of the target gas on the absorption path. Also provided is a photoacoustic sensor for determining a concentration of a target gas in blood of a human or animal animate being.

The present invention relates to a method for determining a concentration of a target gas in blood of a human or animal animate being, a sensor therefor and the use of such a sensor.

The gases oxygen (O₂) and carbon dioxide (CO₂) dissolved in the blood of mammals are specific to the instantaneous state of respiration. They immediately provide information about, for example, an insufficient oxygen supply or a reduced respiratory rate. In the clinical setting, the concentration of oxygen and carbon dioxide is typically determined directly in the exhaled breath via a mask. If such a measurement with the aid of a mask is not possible or is a hindrance in everyday clinical practice, the concentration of the gases can also be determined indirectly transcutaneously, i.e. by means of a sensor above the skin.

In the transcutaneous, non-invasive measurement methods known from the prior art, compact electrodes are stuck on the skin and the gas content is recorded electrochemically. The calibration interval for electrochemical sensors is at most in the range of up to 12 hours, as the electrodes drift over a longer period of time. For calibration, the sensor has to be removed and replaced by another one, and then recalibrated externally.

Sensors that measure the CO₂ concentration in the blood transcutaneously, non-invasively using a non-dispersive infrared (NDIR) sensor are still in the research and development stage. However, the costs of such NDIR sensors are comparatively high and, without a reference channel, NDIR sensors are also susceptible to drift and must be recalibrated at regular, comparatively short time intervals.

Compared to the prior art, it is therefore an object of the present invention to provide a method and a sensor for determining a concentration of a target gas in the blood of a human or animal animate being which, when in use, allow long calibration intervals. In addition, it is an object of the present invention to provide a method for determining a concentration of a target gas in the blood of animate being, and a sensor therefor, which are less expensive than methods and sensors known in the prior art.

At least one of the aforementioned objects is solved by a method for determining a concentration of a target gas in the blood of a human or animal animate being according to independent claim 1. For this purpose, the method according to the invention comprises the steps: Generating electromagnetic excitation radiation having a carrier frequency, wherein the carrier frequency is selected such that the target gas and an absorbing gas absorb the excitation radiation, modulating an amplitude or the carrier frequency of the excitation radiation with a modulation frequency, illuminating an absorption path superficial to a skin of the animate being with the excitation radiation, generating a sound wave by an absorption of the excitation radiation in the absorbing gas, and detecting at least one of an amplitude and a phase of the sound wave as a measure of a concentration of the target gas on the absorption path.

The basic idea of the present invention is to transcutaneously detect the concentration of a target gas in the blood of a human or animal animate being using a photoacoustic method. Photoacoustic methods have the potential to perform a sufficiently accurate determination of a concentration of the target gas at low cost and yet with the required accuracy.

In a photoacoustic method, the absorbing gas, in particular a gas mixture containing the absorbing gas, is irradiated with the electromagnetic excitation radiation, wherein the excitation radiation has a frequency of its electromagnetic alternating field, i.e. a carrier frequency, which is equal to an absorption frequency of the absorbing gas. As a result, the absorbing gas absorbs the excitation radiation. This results in a heating of the target gas and thus in a thermal expansion of the absorbing gas or the gas mixture containing the absorbing gas.

So that conclusions can be drawn about the concentration of the sample gas from the illumination of the absorbing gas with the excitation radiation, the excitation radiation is modulated in such a way that the thermal expansion of the sample gas occurs periodically and thus a sound wave is generated within the gas.

In an embodiment of the invention, the modulation frequency is in a range of 200 Hz to 20 kHz.

By detecting the amplitude or the phase of the sound wave generated in this way, conclusions can be drawn about a property of the absorbing gas, in particular about its concentration. The detected amplitude and/or phase of the sound wave serves as a measure of the concentration of the absorbing gas.

As a physiological process, gases from the blood of animals or humans diffuse through the skin. Thus, the immediate vicinity of the skin superficially thereof has a concentration characteristic of the concentration of the target gases in the blood, especially carbon dioxide. From the concentration of the target gas superficial to the skin, it is possible to infer the respective concentration of the target gas in the blood of the animate being.

In an embodiment of the invention, the absorption path has a distance from the skin of the animate being of 5 cm or less, preferably of 2 cm or less, more preferably of 1 cm or less and particularly preferably of 7 mm or less. Due to the underlying physiological diffusion process, the concentration of the target gases from the blood of the animate being becomes less characteristic with increasing distance from the skin of the animate being. It is crucial for the execution of the method according to the invention that the absorption path is arranged superficially from the skin in such a way that the target gas diffuses from the blood into the region of the absorption path.

In principle, it is possible that the target gas absorbs the excitation radiation as an absorbing gas on the absorption path and the resulting sound wave propagating in the target gas is detected. In such an embodiment of the invention, the target gas and the absorbing gas are identical.

Moreover, in such an embodiment, the amplitude of the generated and detected sound wave is directly proportional to the concentration of the target gas in the region of the absorption path and thus also dependent on the concentration of the target gas in the blood of the animate being. A high concentration of target gas on the absorption path leads to a sound wave with a large amplitude. If the concentration decreases, the amplitude of the sound wave decreases.

In an embodiment of the invention, the absorbing gas is enclosed in a sealed detection volume of a detection chamber. The absorption path also passes through an absorption volume outside the detection volume, wherein the target gas diffuses through the skin of the animate being into the absorption volume. In such an embodiment, the absorption path is divided into two parts. On the one hand, it passes through the absorption volume into which the target gas diffuses from the skin of the animate being and, on the other hand, it passes through the closed detection volume with the absorbing gas in the detection chamber. The absorption volume is arranged in front of the detection volume in a beam direction of the excitation radiation.

The detection chamber contains a predetermined and constant concentration of the absorbing gas. At least the amplitude or the phase of the sound wave is detected, which is generated in the detection chamber due to the absorption of the excitation radiation in the absorbing gas.

However, at least the amplitude or the phase of the sound wave depends on the power of the excitation radiation reaching the absorbing gas in the detection chamber. Since the absorption volume is arranged in front of the detection chamber in the beam direction of the excitation radiation, any absorption of the excitation radiation in the absorption volume leads to an attenuation of the power of the excitation radiation reaching the absorbing gas in the detection volume. The associated reduction in the amplitude of the sound wave detected in the detection chamber or change in its phase is then a measure of the concentration of the target gas in the absorption volume.

Such a two-chamber system with an absorption volume separate from the sealed detection chamber can be described as an absorption spectrometer with a detector in the form of the photoacoustic detection chamber. Compared to an absorption spectrometer with a classical photometer detector, this arrangement has a very precisely defined absorption spectrum due to the absorbing gas. This results in a much higher sensor sensitivity and thus also a high signal-to-noise ratio as well as a high drift stability despite the short absorption path compared to a conventional absorption spectrometer.

An advantage of this embodiment is that the absorption by the target gas in the absorption volume leads to a signal reduction or generally change starting from a level defined by the absorbing gas in the detection chamber. Thus, this method is sensitive to small changes in concentration of the target gas in the absorption volume. The amplitude of the sound wave is inversely proportional to the concentration of the target gas in the absorption volume. An embodiment comprising a closed detection chamber separate from the absorption volume (two-chamber system) has a higher signal-to-noise ratio than a single-chamber system.

The absorption volume within the meaning of the present application is understood to be that portion of the sensor which contains the target gas during operation of the sensor and is at least partially illuminated by the excitation radiation. In an embodiment, the absorption volume is delimited by an absorption chamber formed by the housing, wherein the absorption volume can be connected to the skin surface via a diffusion opening.

The total target gas volume of the sensor must be distinguished from the absorption volume. This target gas volume consists of all cavities of the sensor which contain the target gas diffused from the skin during operation of the sensor. This target gas volume includes the volume of the diffusion opening in addition to the absorption volume in the absorption chamber. In operation, the target gas volume is bordered by the inner wall surfaces of the housing and the skin surface of the animate being. Geometrically, the target gas volume is defined by closing the diffusion opening with a flat plate.

The high sensitivity of a sensor and a measurement method based on a two-chamber system allows the length of the absorption path for the electromagnetic excitation radiation in the absorption volume to be very short. In this context, the length of the absorption path within the meaning of the present application is the geometric length of the absorption volume along a beam axis of the electromagnetic excitation radiation.

It is understood that a transparent element is arranged between the absorption volume and the detection chamber, which separates the two regions from each other and allows electromagnetic radiation to pass from the absorption volume into the detection chamber. In an embodiment of the invention, the considered and measured absorption path is perpendicular to a surface of the transparent element in the absorption volume.

In an embodiment of the invention, the absorption volume is further closed off from the source of the electromagnetic excitation radiation by a transparent element. In such an embodiment, the absorption path is geometrically defined by the perpendicular distance between the two, preferably planar, transparent elements.

In an embodiment, the respective transparent element is a, preferably planar, pane, preferably of glass, plastic or a semiconductor material, which is transparent for the electromagnetic excitation radiation used.

In an embodiment of the invention, the length of the absorption path in the absorption volume is 10 mm or less. In a further embodiment of the invention, the length of the absorption path in the absorption volume is 2 mm or less. In a further embodiment of the invention, the length of the absorption path in the absorption volume is 1.5 mm or less. In a further embodiment of the invention, the length of the absorption path in the absorption volume is 1 mm or less. In still another embodiment of the invention, the length of the absorption path in the absorption volume is 0.5 mm or less. In an embodiment of the invention, the length of the absorption path is 0.1 mm or more.

Such a shortened absorption path, in turn, enables the absorption volume to be designed with a value for its volume that is as small as possible. In an embodiment of the invention, the target gas volume is 1 millilitre or less, preferably 100 microlitres or less, more preferably 1 microlitre or less, further preferably 0.5 microlitres or less, and particularly preferably 0.3 microlitres or less. Since the target gas volume comprises, but is not necessarily identical to, the absorption volume, it is understood that the specified upper limits for the target gas volume also represent appropriate upper limits for the absorption volume.

The size of the target gas volume is largely determined by the absorption volume. The target gas volume determines the response time, i.e. the time constant which the sensor requires to enable a reliable measurement reflecting the arterial gas concentration. Only when an equilibrium has been reached to the effect that no further target gas diffuses through the skin into the target gas volume of the sensor, does the concentration of the target gas in the absorption volume represent the quantity to be measured. If the target gas volume of the sensor is now reduced, the time constant, which dominates the duration of the measurement, is also reduced.

The dimension of the absorption volume defined by the absorption path contributes significantly to the reduction of the absorption volume and thus the target gas volume of the sensor. However, a short length of the absorption path not only reduces this dimension of the absorption volume, but it also enables the other dimensions of the absorption volume to be reduced. While the length of the absorption path is limited by the sensitivity of the sensor and the detection limit to be achieved, the other dimensions of the absorption volume are determined by the requirements of the optical path of the electromagnetic excitation radiation.

If the absorption path is kept short, then in principle comparatively more power of the excitation radiation reaches the detection chamber. Therefore, the cross-section of the absorption volume in the absorption chamber can then also be reduced in a direction perpendicular to the absorption path and thus the beam cross-section of the excitation radiation. The loss of power that may be associated with this is manageable.

Considered now is an embodiment of the invention in which the absorption chamber defining the absorption volume is cuboidal or trapezoidal in shape. The width and height of the absorption volume are then each measured perpendicular to the absorption path. In one embodiment of the invention, the width of the absorption volume is thereby 10 mm or less, preferably 5 mm or less, further preferably 2 mm or less and particularly preferably 1 mm or less. In an embodiment of the invention, the height of the absorption volume is 2 mm or less, preferably 1 mm or less and more preferably 0.5 mm or less.

In an embodiment having a substantially circular or oval cross-section of the absorption chamber perpendicular to the absorption path, the diameter of this cross-sectional area is 10 mm or less, preferably 5 mm or less, more preferably 2 mm or less and particularly preferably 1 mm or less.

In an embodiment of the invention, the absorption volume is closed off from the source of the electromagnetic excitation radiation by a transparent element and the source is arranged outside the absorption volume. The electromagnetic excitation radiation is irradiated into the absorption volume through the transparent element. It is important to prevent the excitation radiation from being absorbed before it passes through the transparent element into the absorption volume, thereby falsifying the measurement result.

In an embodiment, this is implemented in that an exit facet or exit surface of the source for the radiation sits directly on the transparent element bordering the absorption volume. The arrangement of the exit facet or the exit surface of the source for the excitation radiation on the transparent element also has the advantage that, due to the reduced distance between the source and the absorption volume, a larger solid angle of the excitation radiation emerging from the source reaches the absorption volume. This is despite the small cross-section of the absorption volume perpendicular to the absorption path.

In an alternative embodiment, the source is arranged in a source chamber sealed off from the environment, but also from the absorption volume, wherein the source chamber does not contain the target gas or a gas with identical or similar absorption. In an embodiment of the invention, the source chamber is evacuated or contains a gas other than the target gas and the absorbing gas.

In an embodiment of the invention, the transparent elements seal the absorption volume together with the rest of the housing from the environment and together with the housing form the absorption chamber. In an embodiment of the invention, the diffusion opening provides the only access for gas into the absorption volume.

Also, the detection chamber containing the absorbing gas defines a portion of the absorption path for the excitation radiation. In an embodiment of the invention, the length of this section of the absorption path in the detection chamber is defined perpendicular to the planar surface of the transparent element separating the detection chamber from the absorption volume. In an embodiment of the invention, the length of the section of the absorption path in the detection chamber in the beam direction of the excitation radiation is approximately equal to the average absorption length of the absorbing gas. Here, the absorption length of the target gas within the meaning of the present application is that distance in the target gas after which the initially incident excitation radiation has been absorbed to 1/e. Therefore, in an embodiment of the invention, if the absorbing gas is CO₂, the length of the section of the absorption path in the detection chamber is 0.2 mm.

The endeavour to minimise the target gas volume means that the inlet opening into the absorption volume, i.e. the diffusion opening for the target gas, must also be made as small as possible. In an embodiment of the invention, the diffusion opening has a cylindrical inner wall surface with a diameter of less than 50% of the length of the absorption path in the absorption volume, preferably with a diameter of 30% of the length of the absorption path in the absorption volume or less and particularly preferred with a diameter of 10% of the length of the absorption path in the absorption volume or less.

However, minimising the cross-sectional area of the diffusion aperture runs counter to the requirement to cover as large an area as possible of the skin surfaces of the animate being. The larger the area section of the skin included by the sensor in collecting the target gas, the more target gas is available for measurement and the faster is the measurement process. Therefore, in an embodiment of the invention, the sensor comprises a collecting element associated with the target gas volume. The collecting element is in fluid communication with the diffusion opening so that the target gas can diffuse from the skin through the collecting element into the diffusion opening.

In an embodiment of the invention, the collecting element is a sheet of porous, gas-permeable material. In an embodiment of the invention, the collection element is a section of a sheet metal comprising fine gas channels. In this case, the gas channels are drilled or lasered into the sheet metal, for example. In an embodiment, the collecting element is formed by a gas-permeable membrane, wherein collecting gas channels are preferably introduced into the surface of the membrane facing away from the skin.

In an embodiment, the collecting element has an area that is larger than the cross-sectional area of the diffusion opening. In an embodiment, the collecting element has a cross-sectional area through which the target gas can flow that is larger than the cross-sectional area of the diffusion opening.

In an embodiment in which the target gas and the absorbing gas are spatially separated from each other, the target gas and the absorbing gas may be different gases from each other as long as they are absorbing at the carrier frequency of the excitation radiation. However, in another embodiment of the invention, even in such an embodiment, the target gas and the absorbing gas are identical.

In an embodiment in which the absorbing gas is enclosed in a sealed detection chamber and the absorption path further comprises an absorption volume for the target gas, it is sufficient if the absorption path is distanced from the skin of the animate being by 5 cm or less in the region of the absorption volume.

In an embodiment, the method according to the invention further comprises the step of: calculating a concentration of the target gas in the blood of the animate being from the measure of the concentration of the target gas on the absorption path.

In an embodiment of the invention, the calculation of the concentration of the target gas in the blood of the animate being incorporates physiological parameters, such as the diffusion rate of the target gas through the skin, and constructive parameters of the photoacoustic sensor used to carry out the method, such as a value for the volume of the absorption volume.

At least one of the aforementioned objects is also solved by a photoacoustic sensor for determining a content of a target gas in the blood of a human or animal animate being according to the independent claim directed to the photoacoustic sensor. Thereby the photoacoustic sensor comprises: a source configured such that, in an operation of the photoacoustic sensor, the source generates electromagnetic excitation radiation having a carrier frequency, wherein the excitation radiation is amplitude modulated or frequency modulated at a modulation frequency, an absorption volume configured such that, in the operation of the photoacoustic sensor, the target gas diffusing through a skin of the animate being is distributed in the absorption volume, wherein the source and the absorption volume are arranged and configured such that, that, in the operation of the photoacoustic sensor, the electromagnetic excitation radiation of the source illuminates an absorption path within the absorption volume, and a sound detector, wherein the sound detector is arranged and configured such that, in the operation of the photoacoustic sensor, the sound detector detects a sound wave excited by the excitation radiation in an absorbing gas having absorption at the carrier frequency, wherein at least one of amplitude and phase of the sound wave is a measure of a concentration of the target gas in the absorption volume.

Insofar as aspects of the invention are described below with respect to the photoacoustic sensor, these also apply to the method described above for determining a concentration of the target gas, and vice versa. Insofar as the method is carried out with a photoacoustic sensor according to the present invention, the photoacoustic sensor comprises the appropriate means therefor. In particular, embodiments of the photoacoustic sensor are suitable for carrying out the previously described embodiments of the method.

The source of electromagnetic excitation radiation may in an embodiment be an incoherent source, such as a light emitting diode, a glowing emitter or a supercontinuum source, or in another embodiment a coherent source, such as a diode laser.

The choice of the carrier frequency or wavelength of the excitation radiation, and hence the source, depends on which target gas is to be detected from the blood of the animate being. It is understood that the target gas must show absorption at the carrier frequency of the excitation radiation.

The modulation frequency is chosen such that a sound wave which has a frequency equal to the modulation frequency is detectable by means of a conventional sound detector. In an embodiment of the invention, the modulation frequency is in a range of 200 Hz to 20 kHz.

In principle, it is possible to modulate the amplitude of the excitation radiation or its frequency. Crucially, the modulation causes a periodic variation of the absorption and thus the thermal expansion of the absorbing gas.

In an embodiment of the invention, the sound detector is an alternating pressure transducer or an alternating gas flow transducer. An example of an alternating pressure transducer is a microphone, wherein the specific design of the microphone is not important according to the invention. An alternating pressure transducer detects a pressure change in the absorbing gas with which the transducer is in contact.

An alternating gas flow transducer, on the other hand, detects a change in a gas flow of a gas surrounding the transducer. An example of an alternating gas flow transducer is a measuring means based on a change in temperature of a wire, wherein the wire is in the acoustic wave.

In an embodiment of the invention, the photoacoustic sensor also comprises an evaluation device, wherein the evaluation device is operatively connected to the sound detector such that, in operation of the photoacoustic sensor, the evaluation device receives a measurement signal as a measure of the concentration of the target gas in the absorption volume from the sound detector, and wherein the evaluation device is arranged and configured such that that the evaluation device calculates and outputs a concentration of the target gas in the blood of the animate being from the measure for the concentration of the target gas in the absorption volume during operation of the photoacoustic sensor.

In an embodiment of the invention, the photoacoustic sensor comprises a detection chamber, wherein the detection chamber comprises a sealed detection volume separate from the absorption volume, wherein the detection volume of the detection chamber contains the absorbing gas, and wherein the absorbing gas exhibits absorption at the same carrier frequency as the target gas.

In an embodiment of the invention, the absorption volume is enclosed by a housing except for a diffusion opening, wherein the target gas flows into the absorption volume through the diffusion opening during operation of the photoacoustic sensor.

In an embodiment of the invention, the diffusion opening of the housing is sealed with a membrane permeable to the target gas. In this way, contamination of the absorption volume can be prevented during operation of the photoacoustic sensor. Such an embodiment makes the photoacoustic sensor particularly suitable for use in a medical environment.

In an embodiment of the invention, the absorption volume is sealed gas-tight from the environment except for the diffusion opening through the housing.

In an embodiment of the invention, the housing is biocompatible so that it can be brought into contact with the body, in particular the skin, of the animate being without hesitation. Examples of such biocompatible materials include stainless steel, titanium and selected plastics.

In an embodiment of the invention, the housing is sterilisable so that it can be used in a medical environment without hesitation and sterilised after use. In particular, in an embodiment of the invention, the housing is sterilisable in an autoclave.

In an embodiment of the invention, in addition to the absorption volume, the housing also comprises the detection chamber with the detection volume separated from the absorption volume and sealed to the outside.

In a further embodiment of the invention, the housing further comprises the source. In a further embodiment, the housing also comprises the evaluation device.

In an embodiment of the invention, the photoacoustic sensor comprises an interface for connecting the evaluation device to a conventional data network, in particular a LAN or WLAN interface.

In an embodiment, the photoacoustic sensor comprises a display for indicating a concentration of the target gas in the blood of the animate being.

In an embodiment of the invention, the housing has a contact device, wherein the contact device is configured and arranged such that the housing with the contact device can be placed on the skin of the living being such that the diffusion opening faces the skin.

In an embodiment of the invention, the contact device forms a closed circumferential ring around the diffusion opening. Such a ring may be circular, but may also have any other shape, for example rectangular, square, polygonal or oval. It is essential that the ring surrounds the diffusion opening in a closed loop. When this ring is brought into contact with the skin, the target gas can pass through the diffusion opening into the absorption volume, while the rest of the housing seals the absorption volume gas-tightly from the environment.

In an embodiment of the invention, the contact device comprises a sealing element, wherein the sealing element is configured to provide a substantially gas-tight seal between the housing and the skin of the animate being.

In an embodiment of the invention, the sealing element is a seal, in particular a seal made of rubber or caoutchouc, which surrounds the diffusion opening in a closed manner. In an embodiment, the sealing element is a self-adhesive circumferential ring closed around the diffusion opening.

In a further embodiment, the photoacoustic sensor comprises a heating means, wherein the heating means is arranged and configured such that the heating means heats at least the absorption volume or the skin of the animate being in the vicinity of the diffusion opening during operation of the photoacoustic sensor.

Such a heating element provides direct or indirect heating of the skin of the animate being. Heating causes increased blood flow to the skin and surrounding tissue, creating a condition ideal for determining the concentration of the target gas.

In an embodiment of the invention, the heating device is arranged such that the heating device heats the skin locally to a temperature in a range of 40° C. to 45° C., preferably 42° C. Due to the increased blood flow to the tissue when the skin is heated to this temperature range, the transcutaneously measured value for the CO₂ concentration comes very close to the value of the actual CO₂ concentration in an arterial section and the heated skin section.

In another embodiment of the invention, the heating means is arranged to heat an area of the skin underlying the diffusion opening in an operation of the sensor so that the target gas can flow through the heated skin portion into the diffusion opening.

One of the aforementioned objects is also solved by using a photoacoustic sensor according to one of the embodiments described above for determining the concentration of the target gas in the blood of the human or animal embodiment.

In an embodiment, when using the photoacoustic sensor, the housing is placed on the skin of the animate being in such a way that the diffusion opening points in the direction of the skin.

At least one of the aforementioned objects is also solved by a system with a sensor as described above in embodiments thereof, wherein the system is worn on the body of a human or animal being during use. Such systems are also referred to as wearables. Examples of a system worn on the animate being's body during use include a smartwatch, a digital sports watch, an article of clothing, and a “smart care” product for monitoring a vital function.

Further advantages, features and possible applications of the present invention will become apparent from the following description of two embodiments and the accompanying figures. In the figures, the same elements are designated with the identical reference signs.

FIG. 1 is a schematic cross-sectional view through a first embodiment of a photoacoustic sensor for determining a concentration of a target gas in the blood of an animate being.

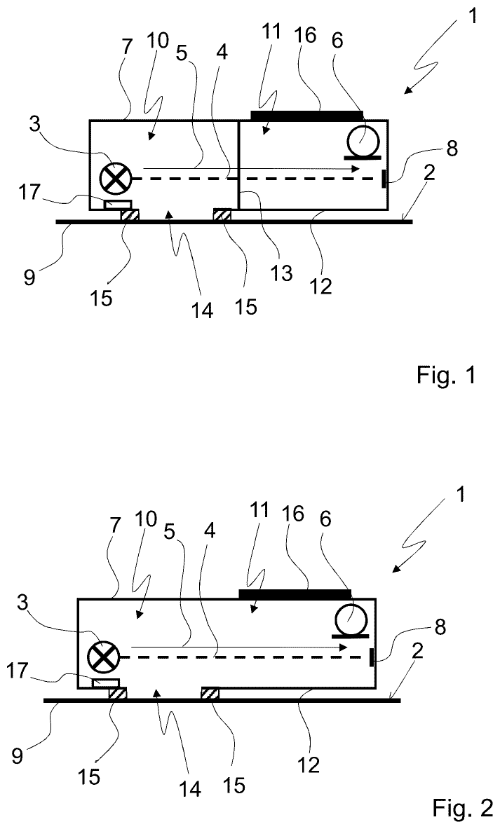

FIG. 2 is a schematic cross-sectional view through a further embodiment of such a photoacoustic sensor.

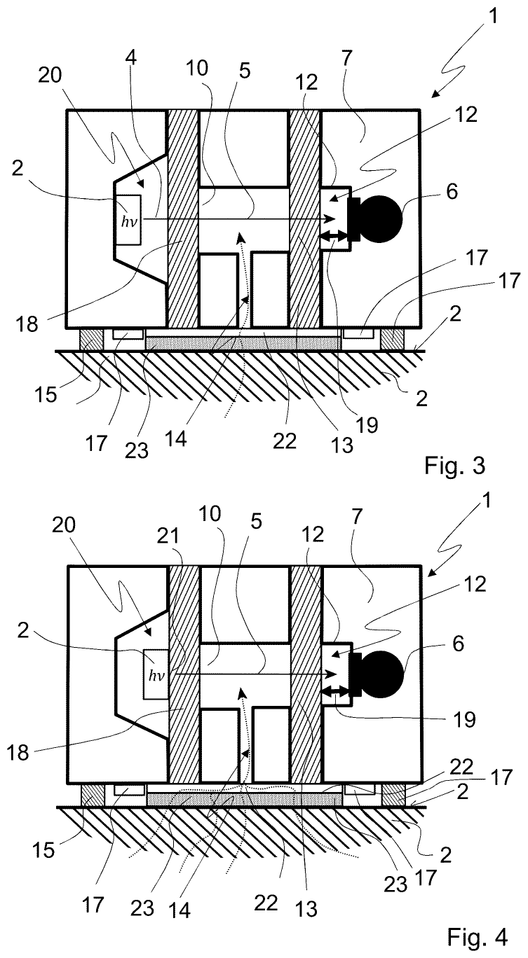

FIG. 3 is a schematic cross-sectional view through yet another embodiment of a photoacoustic sensor for determining a concentration of a target gas in the blood of animate being.

FIG. 4 is a schematic cross-sectional view of a modification of the photoacoustic sensor of FIG. 3.

In the cross-sectional views of FIGS. 1 and 2, the photoacoustic sensors 1 are shown in a lateral cross-sectional view, wherein the photoacoustic sensors are shown during their use for detecting the concentration of a target gas. Therefore, the skin surface 2 of the skin 9 of an animate being is schematically shown. The respective photoacoustic sensor 1 is placed on this skin surface 2.

The embodiments of the photoacoustic sensor 1 shown in FIGS. 1 and 2 are intended to detect the concentration of CO₂ as a target gas within the meaning of the present application. Therefore, the photoacoustic sensors 1 of FIGS. 1 and 2 have a source 3 of electromagnetic excitation radiation with a wavelength, for example, in a range around 4.3 μm. CO₂ absorbs the electromagnetic radiation with this carrier frequency.

The excitation radiation 4 emitted and radiated by the source 3 passes through an absorption path in the photoacoustic sensor 1. A sound wave generated by absorption of the excitation radiation in an absorbing gas is detected with the aid of a microphone 6 as a sound detector within the meaning of the present application.

In both embodiments of FIGS. 1 and 2, the absorbing gas and the target gas are identical in each case. In the embodiment of FIG. 1, an absorbing gas different from the target gas could in principle also be used, as long as it absorbs the excitation radiation at the same carrier frequency or wavelength as the target gas.

The source 3 and the microphone 6 are arranged in a housing 7. To prevent the excitation radiation 4 from irradiating a wall of the housing 7 after passing through the absorption path 5 and generating an additional sound wave there, an absorber 8 is provided in the housing 7 which absorbs the excitation radiation 4 without heating and thus without thermal expansion of the material of the wall 7, the absorber 8 or the gas. In this way, a background or interference signal due to an interaction of the excitation radiation 4 with the housing 7 is avoided. Instead of being absorbed by the absorber 8, the excitation radiation 4 could also be absorbed by the housing 7 or escape from the housing 7 through a window.

In the embodiment of FIG. 1, the housing 7 defines in its interior an absorption volume 10 and a detection volume 11 separated therefrom. The detection volume 11 contains the carbon dioxide in a predetermined, invariable concentration. The detection volume 11 is a sealed volume separated from the absorption volume 10 and from the environment and enclosed in a detection chamber 12. To separate the absorption volume 10 from the detection volume 11, a window 13 transparent to the excitation radiation 4 is provided within the housing 7.

The absorption path 5 is thus divided between the absorption volume 10 and the detection volume 11, wherein the absorption volume 10 is arranged in front of the detection volume 11 in the direction of propagation of the excitation radiation 4.

The excitation radiation 4 from the source 3 comprises an amplitude modulation with a modulation frequency of 700 Hz. By absorbing the excitation radiation in the absorbing gas, a sound wave is generated with a sound frequency which is equal to the modulation frequency.

In the embodiment shown in FIG. 1, the detection chamber 12 forms an acoustic resonator. This resonator has a resonant frequency when it is filled with a gas, in particular a gas mixture containing the absorbing gas. This resonant frequency is equal to the modulation frequency of the excitation radiation 4 and thus to the frequency of the sound wave generated in the absorbing gas. Due to the design of the detection chamber 12 as a resonator, the sound wave experiences an amplitude increase, which in turn improves the signal-to-noise ratio.

If there is no gas absorbing the excitation radiation 4 in the absorption volume 10, a sound wave is generated in the detection volume 11, the amplitude of which is determined by the power of the excitation radiation 4 and the concentration of the CO₂ in the detection volume 11. The amplitude of the sound wave is detected with the aid of the microphone 6.

Since normal ambient air always contains CO₂, when the absorption volume 10 is exposed to ambient air, a certain amount of the excitation radiation 4 is absorbed by the CO₂ contained in the ambient air. The absorption in the absorption volume 10 reduces the power of the excitation radiation 4 available for excitation in the detection volume 11 and the sound wave produced due to the absorption in the detection volume 11 reduces its amplitude.

The housing 7 of the photoacoustic sensor 1 has a diffusion opening 14 in the region of the absorption volume 10, through which gas can flow into the absorption volume 10. In the embodiment shown, the diffusion opening 14 is annularly surrounded by a self-adhesive film 15. On the self-adhesive film 15, the photoacoustic sensor 1 is placed on the skin surface 2 of the skin 9 of the animate being, wherein the self-adhesive film 15 provides a seal of the housing and thus of the absorption volume 10 with respect to the skin surface.

If the target gas, i.e. CO₂, now diffuses from the blood of the animate being through the skin 9 into the region of the diffusion opening 14 as a result of physiological processes, the CO₂ enters the absorption volume 10. There the CO₂ absorbs the excitation radiation 4 and reduces the power of the excitation radiation 4, which enters the detection volume 11. As the concentration of CO₂ diffusing from the blood through the skin 9 into the absorption volume 10 increases, the amplitude of the sound wave detected by the microphone 6 decreases or the phase of the sound wave changes.

In the embodiments shown in FIGS. 1 and 2, the photoacoustic sensor 1 comprises a display 16 with an evaluation device, wherein the evaluation device 16 is connected to the microphone 6. In this way, the evaluation device 16 receives measured values during operation of the photoacoustic sensor 1 and thus a measure of the amplitude of the sound wave from the microphone 6. From the measure of the amplitude of the generated sound wave, the evaluation device 16 can calculate a measure of the concentration of the target gas in the absorption volume 10. Since the concentration of CO₂ in the ambient air can be estimated fairly accurately and the diffusion process as such is known, a measure of the concentration of CO₂ in the blood of the animate being can be determined from the concentration of CO₂ in the absorption volume 10.

The photoacoustic sensor 1 further comprises a heating device 17. The heating device 17 can be used to heat the housing in the vicinity of the diffusion opening 14. Since the housing is in thermal contact with the skin surface 2 via the very thin self-adhesive film 15, the heating device 17 also heats the skin and surrounding tissue during operation of the photoacoustic sensor 1. The heating stimulates the blood flow in this area of the animate being and the concentration measurement of CO₂ in the absorption volume 10 becomes more meaningful for the actual concentration of CO₂ in the blood.

FIG. 2 shows a variant of the embodiment of the photoacoustic sensor 1 of FIG. 1. The photoacoustic sensor 1 has only one absorption volume 10, so that the absorption path 5 lies completely within the absorption volume 10. An additional, separate detection volume does not exist here. In such an embodiment, the amplitude of the sound wave generated by the absorption of the excitation radiation 4 in the CO₂ depends directly on the concentration of the CO₂ in the absorption volume 10. The higher the CO₂ concentration in the absorption volume 10, the greater the amplitude of the sound wave. Such an embodiment is technically simpler and therefore less expensive to implement, but may have a poorer signal-to-noise ratio than the photoacoustic sensor 1 of FIG.

FIGS. 3 and 4 each show a photoacoustic sensor 1 based on a two-chamber arrangement. The principle of such a two-chamber arrangement has already been explained with reference to the embodiment of FIG. 1. In contrast to the embodiment of FIG. 1, the two variants of FIGS. 3 and 4 have an absorption path 5 in the absorption volume 10, which is precisely defined by two plane windows 13, 18.

In embodiments of FIGS. 3 and 4, the absorption volume 10 in the absorption chamber defined by the housing 7 and the windows 13, 18 is as small as possible and the section 19 of the absorption path in the detection chamber 12 is as short as possible.

The reduction of the absorption volume 10 is achieved on the one hand by shortening the absorption path 5 in the absorption chamber. In the embodiments shown in FIGS. 3 and 4, the length of the absorption path 5 is measured along the beam axis of the excitation radiation 4. The length of the section of the absorption path 5 in the absorption chamber is equal to the perpendicular distance between the two windows 13, 18 which delimit the absorption volume 10 along the beam axis. The length of the absorption path 10 in the absorption chamber is only 1.5 mm in both embodiments.

The reduced length of the absorption path 10 in the absorption chamber allows a reduction of the other dimensions of the absorption volume 10, i.e. the cross-section of the absorption volume perpendicular to the absorption path 5. If the absorption path 5 is kept short, then in principle comparatively a large amount of intensity of the electromagnetic radiation 4 passes through the absorption volume 10 into the detection chamber 12. Therefore, the cross-section of the absorption volume and thus the beam cross-section of the electromagnetic excitation radiation can then also be reduced. In the embodiment shown, the absorption volume 10 has a circular cross-section, wherein the cross-sectional area perpendicular to the absorption path 5 has a diameter of only 1 mm.

In addition to the absorption volume 10, the target gas volume of the sensors 1 of FIGS. 3 and 4 also comprises the volume of the diffusion opening 14 and the volume of a collecting element 10. In FIGS. 3 and 4, the target gas volume of the sensors is bordered by the skin surface 2 or a flat plate placed in its place.

In the embodiments shown, the absorption volume 10 is only 0.5 microlitres. The target gas volume is only slightly larger.

The absorption volume 10 is closed off from the source 3 for the electromagnetic excitation radiation 4 by the window 18. The source 3 is thus positioned outside the absorption volume 10. The electromagnetic excitation radiation 4 is irradiated into the absorption volume 10 through the window 18. In the embodiment of FIG. 3, the source 3 is arranged in a source chamber 20 which is sealed off from the surroundings, but also from the absorption volume 10. The source chamber 20 is evacuated so that the excitation radiation 4 does not undergo any absorption that could falsify the measurement result before it passes through the window 18 into the absorption volume 10.

In contrast, in the embodiment of FIG. 4, the exit surface 21 of the light-emitting diode as source 3 is located directly on the window 18, so that there is no gas between the exit facet 21 and the window 18.

The detection chamber 12 with the absorbing gas also forms a further section 19 of the absorption path for the excitation radiation 4. The length of this second section 19 of the absorption path perpendicular to the plane surface of the window 13 is equal to the mean absorption length of the strongest absorption lines of the absorbing gas. In the embodiments of FIGS. 3 and 4, the absorbing gas and the target gas are CO₂. Therefore, the length of the section 19 of the absorption path in the detection chamber 12 is only 0.2 mm.

The diffusion opening 14 provides the only access for gas into the absorption volume 10. Minimising the target gas volume with the absorption volume 10 and the volume of the diffusion opening 14 results in a faster equilibrium of the partial pressures of the target gas in the animate being's body and in the absorption volume 10, thereby reducing the time after which a change in the arterial target gas concentration in the animate being can be reliably detected.

On the other hand, for effective collection of the target gas, it is important to include as large an area of the skin surface 2 as possible in the collection of the target gas, so that as much target gas as possible flows into the absorption volume 10.

Therefore, in embodiments of FIGS. 3 and 4, the sensor 1 comprises a collecting element 22 that effectively increases the target gas collecting area of the sensor without increasing the target gas volume of the sensor to the same extent. The collecting element 22 is in fluid communication with the diffusion opening 14.

In the embodiments shown, the collecting element 22 is a thin porous and therefore gas permeable sheet of plastic. In the drawing of FIG. 4, the collecting action of the collecting element 22 is schematically indicated. The collecting element 22 is covered towards the skin surface 2 with a membrane 23 permeable to the target gas.

For the purposes of the original disclosure, it is pointed out that all features as they become apparent to a person skilled in the art from the present description, the drawings and the claims, even if they have been specifically described only in connection with certain further features, can be combined both individually and in any desired combinations with other of the features or groups of features disclosed herein, unless this has been expressly excluded or technical circumstances render such combinations impossible or pointless. A comprehensive, explicit description of all conceivable combinations of features is omitted here only for the sake of brevity and readability of the description.

While the invention has been illustrated and described in detail in the drawings and the foregoing description, this illustration and description are merely exemplary and are not intended to limit the scope of protection as defined by the claims. The invention is not limited to the embodiments disclosed.

Variations of the disclosed embodiments will be apparent to those skilled in the art from the drawings, description and appended claims. In the claims, the word “comprising” does not exclude other elements or steps, and the indefinite article “one” or “a” does not exclude a plurality. The mere fact that certain features are claimed in different claims does not exclude their combination. Reference signs in the claims are not intended to limit the scope of protection.

### LIST OF REFERENCE SIGNS

1 photoacoustic sensor
2 skin surface
3 source
4 excitation radiation
absorption path
6 microphone
7 housing
8 absorber
9 skin
10 absorption volume
11 detection volume
12 detection chamber
13, 18 window
14 diffusion opening
15 self-adhesive film
16 evaluation device
17 heating device
19 section of the absorption section in the detection chamber
20 source chamber
21 outlet surface of source 3
22 collecting element
23 membrane

1. A method for determining a concentration of a target gas in blood of a human or animal animate being, comprising the steps of:
generating electromagnetic excitation radiation having a carrier frequency, wherein the carrier frequency is selected such that the target gas and an absorbing gas absorb the excitation radiation,
modulating an amplitude or the carrier frequency of the excitation radiation with a modulation frequency,
illuminating an absorption path superficially of a skin of the animate being with the excitation radiation,
generating a sound wave by an absorption of the excitation radiation in the absorption gas, and
detecting at least one of an amplitude and a phase of the sound wave as a measure of a concentration of the target gas on the absorption path.

2. The method according to claim 1, wherein the absorption path is distanced from the skin of the animate being by 5 cm or less.

3. The method according to claim 1, wherein the absorption gas is enclosed in a sealed detection chamber, wherein the absorption path passes through an absorption volume outside the detection chamber and in a beam direction of the excitation radiation in front of the detection chamber, wherein the target gas diffuses through the skin of the animate being into the absorption volume.

4. A photoacoustic sensor for determining a content of a target gas in blood of a human or animal animate being, wherein the photoacoustic sensor comprises:
a source configured such that in an operation of the photoacoustic sensor the source generates electromagnetic excitation radiation having a carrier frequency, wherein the excitation radiation is amplitude modulated or frequency modulated at a modulation frequency,
an absorption volume configured such that, in the operation of the photoacoustic sensor, the target gas diffused through a skin of the animate being is distributed in the absorption volume, wherein the source and the absorption volume are arranged and configured such that, in the operation of the photoacoustic sensor, the electromagnetic excitation radiation of the source illuminates an absorption path within the absorption volume, and
a sound detector, wherein the sound detector is configured and arranged such that in the operation of the photoacoustic sensor the sound detector detects a sound wave excited by the excitation radiation in an absorbing gas, which absorbing gas comprises an absorption at the carrier frequency, wherein at least one of amplitude and phase of the sound wave is a measure of a concentration of the target gas in the absorption volume.

5. The photoacoustic sensor according to claim 4, wherein the photoacoustic sensor comprises a detection chamber,
wherein the detection chamber comprises a sealed detection volume separate from the absorption volume,
wherein the detection chamber contains the absorption gas, and
wherein the absorbing gas exhibits absorption at the same carrier frequency as the target gas.

6. The photoacoustic sensor according to claim 5, wherein the length of the absorption path in the absorption volume is 10 mm or less.

7. The photoacoustic sensor according to claim 5, wherein the 1 millilitre or less.

8. The photoacoustic sensor according to claim 5, wherein a length of the section of the absorption path in the detection chamber is approximately equal to the average absorption length at the strongest absorption line of the absorption gas.

9. The photoacoustic sensor according to claim 4, wherein the absorption volume is closed off at least in sections by a housing, wherein the housing has a diffusion opening through which the target gas flows into the absorption volume during operation of the photoacoustic sensor.

10. The photoacoustic sensor according to claim 9, wherein the diffusion opening comprises a cylindrical inner wall surface with a diameter of less than 50% of the length of the absorption path in the absorption volume.

11. The photoacoustic sensor according to claim 9, wherein the photoacoustic sensor comprises a collecting element, wherein the collecting element is in fluid communication with the diffusion opening such that the target gas can diffuse from the skin through the collecting element into the diffusion opening, and wherein the collecting element has a cross-sectional area through which the target gas can flow, which cross-sectional area is larger than a cross-sectional area of the diffusion opening.

12. The photoacoustic sensor according to claim 4, wherein the housing comprises a contact device, wherein the contact device is configured and arranged such that the housing with the contact device can be placed on the skin of the animate being such that the diffusion opening points towards the skin, wherein the contact device preferably forms a closed circumferential ring around the diffusion opening and wherein the contact device preferably comprises a sealing element, wherein the sealing element is configured in such a way that the sealing element can be used to provide an essentially gas-tight seal between the housing and the skin of the animate being.

13. The photoacoustic sensor according to claim 4, wherein the photoacoustic sensor comprises a heating device, wherein the heating device is arranged and configured such that the heating device, in operation of the photoacoustic sensor, heats at least the absorption volume or the skin of the animate being in a vicinity of the diffusion opening.

14. The photoacoustic sensor according to claim 4, wherein the source is a thermal source or a light emitting diode.

15. A system comprising the photoacoustic sensor according to claim 4, wherein the system is wearable on a body of the human or animal animate being during use.
